(19) United States
(12) Patent Application Publication
(10) Pub. No.: US 2021/0338052 A1
(43) Pub. Date: Nov. 4, 2021

(54) PORTABLE ENDOSCOPE WITH STEERABLE CANNULA

(71) Applicant: UroViu Corp., Bellevue, WA (US)

(72) Inventors: Xiaolong Ouyang, Bellevue, WA (US); Chih-Yu TING, New Taipei City (TW); Shih-Ping WANG, Los Altos, CA (US)

(21) Appl. No.: 17/370,575

(22) Filed: Jul. 8, 2021

Related U.S. Application Data
(63) Continuation-in-part of application No. 17/145,466, filed on Jan. 11, 2021.
Application No. 17/145,466 is a division of application No. 16/447,251, filed on Jun. 20, 2019, now Pat. No. 11,013,396.

(51) Int. Cl. A61B 1/005; A61B 1/00

(52) U.S. Cl. CPC A61B 1/0052; A61B 1/0057; A61B 1/00103; A61B 1/307; A61B 1/00144; A61B 1/00048; A61B 1/00128

(57) ABSTRACT

A handheld endoscope has a disposable, single-use portion that includes a fluid hub, cannula, distal tip and is steerable by the operator though actuation of one or two levers. The endoscope also includes multiple-use portion that has a handle and display module. The distal tip includes LED illumination and an imaging module that feeds live video to the display module that is rotatable to allow viewing by the operator and others. The single-use and multiple-use portions mate and un-mate with each other via physically separated mechanical and electrical connectors. The component of the endoscope can be supplied to users in different combinations of pre-assembled configurations, some in a sterile package. The single use portion can include grasper device that can be actuated by an operator using an actuation tab.

### REFERENCE TO RELATED APPLICATIONS

This patent application is a continuation-in-part of parent U.S. patent application Ser. No. 17/145,466 filed Jan. 12, 2021, which in turn is a divisional of application Ser. No. 16/447,251 filed Jun. 20, 2019, now U.S. Pat. No. 11,013,396 issued on May 25, 2021. This application claims the benefit and incorporates by reference each of said application Ser. Nos. 17/145,466 and 16/447,251 and each of the 10 following provisional patent applications:

U.S. Prov. Ser. No. 63/032,784 filed May 30,02020;

U.S. Prov. Ser. No. 63/035,570 filed Jun. 5, 2020;

U.S. Prov. Ser. No. 63/037,792 filed Jun. 11, 2020;

U.S. Prov. Ser. No. 63/038,118 filed Jun. 11, 2020;

U.S. Prov. Ser. No. 63/045,288 filed Jun. 29, 2020.

Said U.S. patent application Ser. No. 16/447,251 claims the benefit of and incorporates by reference each of the following provisional applications:

U.S. Prov. Ser. No. 62/842,297 filed May 2, 2019;

U.S. Prov. Ser. No. 62/825,948 filed Mar. 29, 2019;

U.S. Prov. Ser. No. 62/821,536 filed Mar. 21, 2019;

U.S. Prov. Ser. No. 62/821,430 filed Mar. 20, 2019;

U.S. Prov. Ser. No. 62/797,235 filed Jan. 26, 2019;

U.S. Prov. Ser. No. 62/796,346 filed Jan. 24, 2019;

U.S. Prov. Ser. No. 62/795,042 filed Jan. 22, 2019;

U.S. Prov. Ser. No. 62/791,045 filed Jan. 11, 2019; and

U.S. Prov. Ser. No. 62/729,061 filed Sep. 10, 2018.

This patent application incorporates by reference each of the following provisional and non-provisional patent applications and issued patent(s):

U.S. Pat. No. 9,895,048 issued Feb. 20, 2018;

U.S. Pat. No. 10,278,563 issued May 7, 2019;

U.S. Pat. No. 10,292,571 issued May 21, 2019;

U.S. Pat. No. 10,524,636 issued Jan. 7, 2020;

U.S. Pat. No. 10,874,287 issued Dec. 29, 2020;

U.S. Pat. No. 10,869,592 issued Dec. 22, 2020;

U.S. Pat. No. 10,918,268 issued Feb. 16, 2021;

U.S. Pat. No. 11,013,396 issued May 25, 2021;

U.S. Ser. No. 16/407,028 filed May 8, 2019;

Int'l Pat. App. No. PCT/US18/14880 filed Jan. 23, 2018;

Int'l Pat. App. No. PCT/US16/65396 filed Dec. 7, 2016;

Int'l Pat. App. No. PCT/US16/18670 filed Feb. 19, 2016;

U.S. Prov. Ser. No. 63/009,389 filed Apr. 13, 2020;

U.S. Prov. Ser. No. 62/647,454 filed Mar. 23, 2018;

U.S. Prov. Ser. No. 62/634,854 filed Feb. 24, 2018;

U.S. Prov. Ser. No. 62/587,038 filed Nov. 16, 2017;

U.S. Prov. Ser. No. 62/551,264 filed Aug. 29, 2017;

U.S. Prov. Ser. No. 62/452,883 filed Jan. 31, 2017;

U.S. Prov. Ser. No. 62/449,257 filed Jan. 23, 2017;

U.S. Prov. Ser. No. 62/443,769 filed Jan. 8, 2017;

U.S. Prov. Ser. No. 62/416,403 filed Nov. 2, 2016;

U.S. Prov. Ser. No. 62/405,930 filed Oct. 9, 2016;

U.S. Prov. Ser. No. 62/375,814 filed Aug. 16, 2016;

U.S. Prov. Ser. No. 62/362,643 filed Jul. 15, 2016;

U.S. Prov. Ser. No. 62/339,810 filed May 21, 2016;

U.S. Prov. Ser. No. 62/299,453 filed Feb. 24, 2016

U.S. Prov. Ser. No. 62/287,901 filed Jan. 28, 2016;

U.S. Prov. Ser. No. 62/279,784 filed Jan. 17, 2016;

U.S. Prov. Ser. No. 62/275,241 filed Jan. 6, 2016;

U.S. Prov. Ser. No. 62/275,222 filed Jan. 5, 2016;

U.S. Prov. Ser. No. 62/259,991 filed Nov. 25, 2015;

U.S. Prov. Ser. No. 62/254,718 filed Nov. 13, 2015;

U.S. Prov. Ser. No. 62/139,754 filed Mar. 29, 2015;

U.S. Prov. Ser. No. 62/120,316 filed Feb. 24, 2015; and

U.S. Prov. Ser. No. 62/119,521 filed Feb. 23, 2015.

### FIELD

This patent specification generally relates to a medical device for use in tissue examinations and endoscopic surgery such as in urology or similar fields. More particularly, some embodiments relate to a portable, handheld, low-cost surgical endoscope device having a single-use cannula with a tip that is conveniently and effectively steerable and includes an imaging module , and additional components that can be single-use or can be multiple-use and can have different cannulas attached thereto.

### BACKGROUND

Conventional endoscopy, or direct vision, used to examine the interior of a hollow organ or cavity of the body, uses a complex lens system for transmitting the image from the distal tip of the endoscope to a viewer. The lens system is typically a relay lens system in the case of rigid endoscopes or a bundle of fiber optics or an objective lens system in the case of flexible endoscopes. In the case of both rigid and flexible conventional endoscopes, the lens or fiber optic system is relatively expensive and is intended to be re-used many times. Therefore, stringent decontamination and disinfection procedures need to be carried out after each use.

In surgical procedures where a needle is used to inject fluid such as a drug into the patient's tissues, a long injection needle is inserted into the working channel of the endoscope. In such procedures, it is common to use two or more operators to carry out the surgical procedure: one to operate the endoscope and another to operate the needle assembly and syringe. It is common for there to be a physical separation between a display screen (e.g. mounted overhead), the endoscope (into the patient), and/or the syringe used to administer the drug. In such cases an operator or clinician has to look up to the display screen and cannot simultaneously view the scope handle and the syringe. Furthermore, the separate needle assembly, which is often long and somewhat cumbersome, needs to be threaded through the working channel of the endoscope and substantial manual dexterity may be required to control the jabbing and injection process. In some procedures, endoscopes with deflectable distal portions may be suggested, for example as discussed in U.S. Pat. Nos. 8,834,357 and 8,845,522.

Disposable endoscopy is an emerging category of endoscopic instruments. In some cases the manufacture of endoscopes can be made inexpensive enough to be used on a single patient only. Disposable or single-use endoscopy lessens the risk of cross-contamination and hospital acquired diseases. Partially disposable endoscopy systems are discussed in U.S. Pat. Nos. 9,895,048, 10,278,563, and 10,292,571 cited above. The subject matter described or claimed in this patent specification is not limited to embodiments that solve any specific disadvantages or that operate only in environments such as those described above. Rather, the above background is only provided to illustrate one exemplary technology area where some embodiments described herein may be practiced.

### SUMMARY

Some embodiments that are particularly suitable for fields such as urology although the equipment and methods disclosed in this patent application can be used in other medical fields as well.

According to some embodiments, an endoscope for procedure in a patient comprises: a cannula having a bendable distal portion and an imaging module distal from said bendable portion; a fluid hub, a steering hub, a handle, and a steering lever, wherein: said fluid hub has a distal end configured to couple with a proximal end of said cannula; said steering hub has a distal end configured to couple with a proximal end of said fluid hub; said handle has a distal end configured to couple with a proximal end of said fluid hub; and said steering lever is operatively coupled to said bendable portion of the cannula and is configured to respond to manual manipulation of the lever to bend said bendable portion of the canula in at least two differed angular direction and to a selected degree in each of said angular directions; wherein said cannula, fluid hub, steering hub, and handle are in a selected one of first through fourth configurations as supplied to a user wherein: (i) in the first configuration said cannula, fluid hub, and steering hub are assembled connected to each other as a first single-use portion and said first single use portion is in a first sterile pouch but said handle comprises a first multiple-use portion that is outside the first sterile pouch; (ii) in the second configuration said cannula and fluid hub are assembled connected to each other in a second single-use portion and said second single use portion is in a second sterile pouch but said steering hub and said handle are assembled connected to each other as a second multiple-use portion that is outside the second sterile pouch; (iii) in the third configuration said cannula comprises a third single-use portion and said third single use portion is in a third sterile pouch but said fluid hub, steering hub and handle are assembled together as a third multiple-use portion that is outside the third sterile pouch; and (iv) in the fourth configuration, said cannula, fluid hub, steering hub, and handle are separate and unconnected to each other as supplied to a user; and wherein said cannula, fluid hub, steering hub, and handle are configured for assembly into said endoscope for a procedure in a patient.

According to some embodiments, the endoscope further includes one or more of the following features: (a) said cannula, fluid hub, steering hub, and handle are supplied to a user in said first configuration; (b) said cannula, fluid hub, steering hub, and handle are supplied to a user in said second configuration; (c) said cannula, fluid hub, steering hub, and handle are supplied to a user in said third configuration; (d) said cannula, fluid hub, steering hub, and handle are supplied to a user in said fourth configuration; (e) said cannula is configured for rotation relative to the handle when said cannula, fluid hub, steering hub, and handle are assembled with each other for form said endoscope; (f) the endoscope further includes a display integrally mounted on said handle and configured to display images taken with said imaging module and said handle comprises internal circuits configured to process image data supplied by said imaging module into images displayed on said display; (g) the endoscope further includes a flexible and stretchable sterile robe into which said display and handle fit, said robe including a transparent window matching a screen of said display; (h) the endoscope further includes a processing and display assembly remote from said handle and electrically coupled therewith via a cable to receive image data from said imaging module and process said imaging date into images for display and display said images; (i) the endoscope further includes a display remote from said handle and electrically coupled therewith via a cable to receive image data from said imaging module; (j) said steering hub and fluid hub are configured for rotation relative to each other about a longitudinal axis of said cannula when the cannula, fluid hub, steering hub, and handle are assembled to form said endoscope; (k) the endoscope further comprises a display supported by and mounted on said handle and having a screen to display images provided by said imaging module, and a sterile robe configured to fit over said handle and display and having a transparent window covering said screen; (I) said bendable portion of the cannula is configured to bend in one of said angular direction through a maximum that is less than a maximum through which said bendable portion is configured to bend in the other of said angular directions; and (m) said steering hub for rotation comprises an internal wheel and further including cables secured at one end to said wheel and at the other end to said bendable portion of the cannula.

According to some embodiments, an integrated, single use endoscope comprises: a cannula having at least one lumen extending along a length of the cannula, a bendable distal portion, and an electronic imaging module at a distal portion of the cannula; a fluid hub permanently connected operatively to a proximal portion of the cannula and having at least one port in fluid flow communication with said lumen; a handle permanently connected operatively to a proximal portion of the fluid hub; a steering control operatively connected to said bendable portion of the cannula and manually operable to selectively bend said bendable portion through selected angles in at least two angular directions; and an electronic port operative coupled with said imaging module and configured to receive power and commands from a location remote from the endoscope and to provide to said remote location image date generated by said imaging module; wherein processing of said image date into images for display takes place primarily outside of said endoscope, and said endoscope is configured for only a single use in a patient procedure.

According to some embodiments, the endoscope of the immediately preceding paragraph includes one or more of the following features: (a) said endoscope is operatively coupled with said remote location via a cable external to the endoscope; and (b) said endoscope is operatively coupled with said remote location wirelessly.

According to some embodiments, a method comprises: providing a cannula having an internal lumen and a bendable distal portion with an imaging module distal from said bendable portion, a fluid hub proximal to the cannula, a steering hub proximal to the fluid hub, a handle proximal to the steering hub, an image display integral with said handle, and a steering lever: selectively bending the bendable portion of the cannula in at least two angular directions and though selected angles in said angular directions by manually operating said steering lever; selectively rotating at least one of the cannula and the fluid hub relative to the handle about a long axis of the cannula; and producing image data with said imaging module, supplying the image data to said handle and display for processing the image data into display images and displaying said images.

According to some embodiments of the method: said rotating comprises rotating the cannula relative to the housing, and/or said rotating comprises rotating both the cannula and the fluid hub relative to the housing

As used herein, the grammatical conjunctions “and”, “or” and “and/or” are all intended to indicate that one or more of the cases, object or subjects they connect may occur or be present. In this way, as used herein the term “or” in all cases indicates an “inclusive or” meaning rather than an “exclusive or” meaning.

As used herein the terms “surgical” or “surgery” refer to any physical intervention on a patient's tissues, and does not necessarily involve cutting a patient's tissues or closure of a previously sustained wound.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims. The subject matter hereof will be described and explained with additional specificity and detail through the use of the accompanying drawings in which:

FIGS. 1A-1C are perspective views of a portable endoscope having a slim disposable steerable cannula, according to some embodiments;

FIG. 2 is a side view of a slim disposable steerable cannula, according to some embodiments;

FIGS. 3A, 3B and 3C are right side, top and left side views, respectively, of a disposable steerable cannula, according to some embodiments;

FIGS. 4A and 4B show further detail of an steering actuation hub of a portable endoscope having a slim disposable steerable cannula, according to some embodiments;

FIG. 5 is an exploded view illustrating various components of a disposable portion 104, according to some embodiments;

FIGS. 6A and 6B are a side view and perspective view and two cross sections, respectively, illustrating further detail of a distal portion of an endoscope according to some embodiments;

FIGS. 6C-6H are cross sections illustrating further details of a distal portion of an endoscope according to some embodiments;,

FIG. 7 is a side view of a slim disposable steerable cannula, according to some embodiments;

FIG. 8 shows further detail of a steering actuation hub of a portable endoscope having a slim disposable steerable cannula, according to some embodiments;

FIGS. 9A and 9B are a right side view a perspective view, respectively, of a handheld surgical endoscope having an integrated grasping tool, according to some embodiments;

FIGS. 10A and 10B are perspective views of a distal tip 912 and show aspects of the grasper actuation, according to some embodiments;

FIGS. 11A and 11B are perspective views showing aspects of grasper actuation for a handheld surgical endoscope, according to some embodiments;

FIG. 12 is a side view of an endoscope with a rotatable cannula, according to some embodiments.

FIG. 13 is a perspective view of an endoscope divided into three subassemblies that can be supplied assembled in different combinations with each other, or separately, according to some embodiments.

FIG. 14 is a perspective view of an endoscope supplied in two subassemblies, according to some embodiments.

FIG. 15 schematically illustrates an endoscope supplied in a different set of two subassemblies, according to some embodiments.

FIG. 16 schematically illustrates an endoscope supplied in yet another different set of two subassemblies, according to some embodiments.

FIG. 17 is a perspective view of a proximal side of a handle with a modified electrical connector socket, according to some embodiments.

FIG. 18 is a partly perspective and partly front view of an endoscope that can connect to a remote signal processor and display via a cable or through a wireless connection, according to some embodiments.

FIG. 19 is a perspective view of an endoscope with modified electrical connectors that can be used in place of the electrical connectors in any of the other illustrated endoscopes, in accordance with some embodiments.

FIG. 20 is a perspective view of an endoscope in a sterile robe for use in sterile environments, according to some embodiments.

FIG. 21 is a perspective view of a sterile robe for an endoscope, according to some embodiments.

### DETAILED DESCRIPTION

A detailed description of examples of preferred embodiments is provided below. While several embodiments are described, it should be understood that the new subject matter described in this patent specification is not limited to any one embodiment or combination of embodiments described herein, but instead encompasses numerous alternatives, modifications, and equivalents. In addition, while numerous specific details are set forth in the following description in order to provide a thorough understanding, some embodiments can be practiced without some or all of these details. Moreover, for the purpose of clarity, certain technical material that is known in the related art has not been described in detail in order to avoid unnecessarily obscuring the new subject matter described herein. It should be clear that individual features of one or several of the specific embodiments described herein can be used in combination with features of other described embodiments or with other features. Further, like reference numbers and designations in the various drawings indicate like elements.

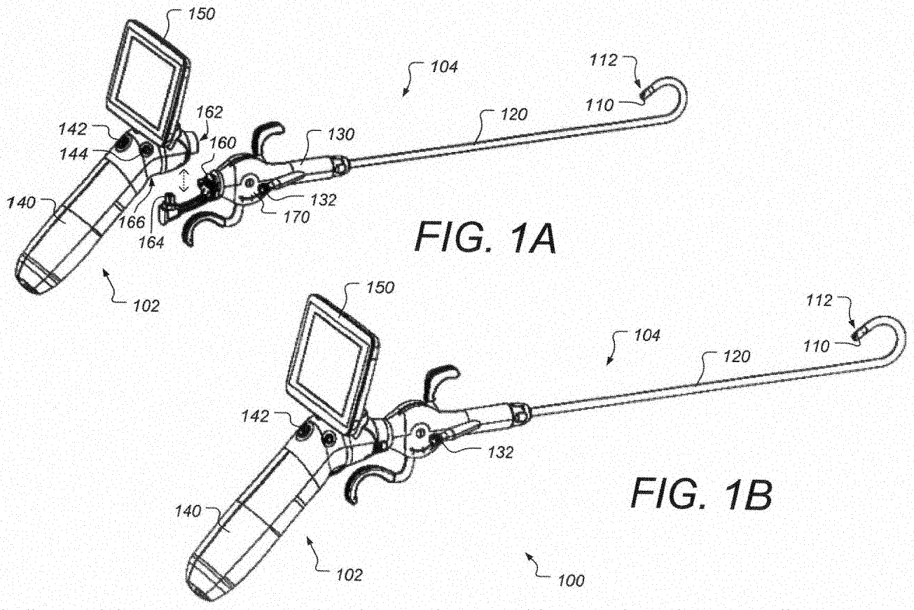

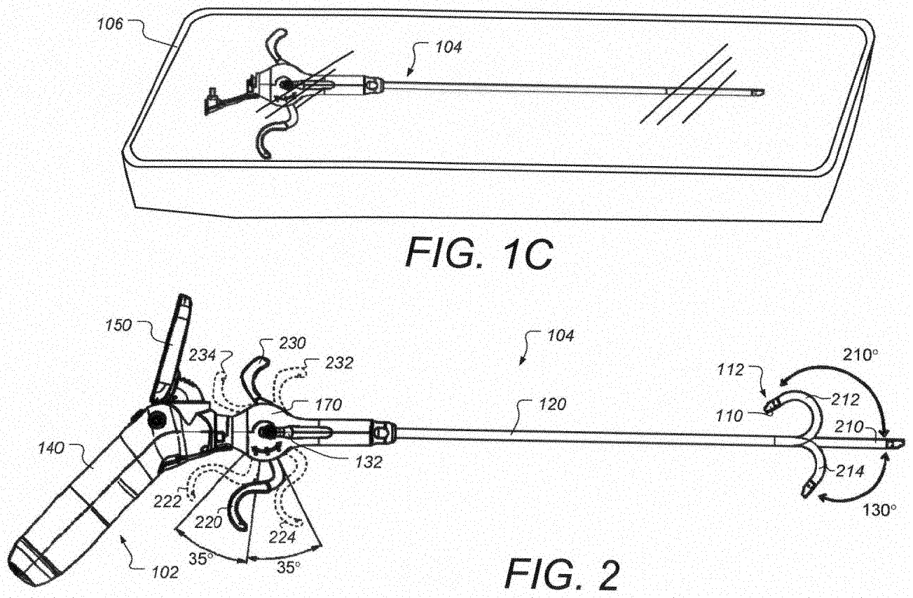

FIGS. 1A-1C are perspective views of a portable endoscope having a slim disposable steerable cannula, according to some embodiments. FIGS. 1A and 1B illustrate aspects of attachment and detachment of the single-use and reusable portions of handheld endoscope 100, according to some embodiments. The single-use portion 104 and reusable portion 102 attach mechanically primarily via mating mechanical connectors 160 and 162 as shown by the dotted arrow in FIG. 1A. Electrical connection is made via separate mating electrical connectors 164 and 166. In this example the two portions 102 and 104 are attached mechanically via translation vertically towards each other. Note that electrical connector 164 and mechanical connector 160 are both separated from the fluid hub 130, and are separated from each other by a distance of several cm, e.g., 5 cm or more. This separation allows for easy and effective, yet simple and inexpensive, fluid sealing to prevent fluid from hub 130, and any fluid from steering actuation hub 170 from penetrating internally towards connectors 160 and 164 and also allows some protection against any exterior fluid, for example from fluid port 132, from reaching and possibly compromising electrical connectors 164 and 166. The physical separation of the fluid hub 130 and the mechanical and electrical connectors 160 and 164 also provide additional assurance against accidental contamination from fluid hub 130 to the re-usable portion 102. For even greater protection from fluid contamination, fluid hub 130 can be made longer and ports For further details regarding the physical separation and associated benefits, see said U.S Pat No. 9,895,048.

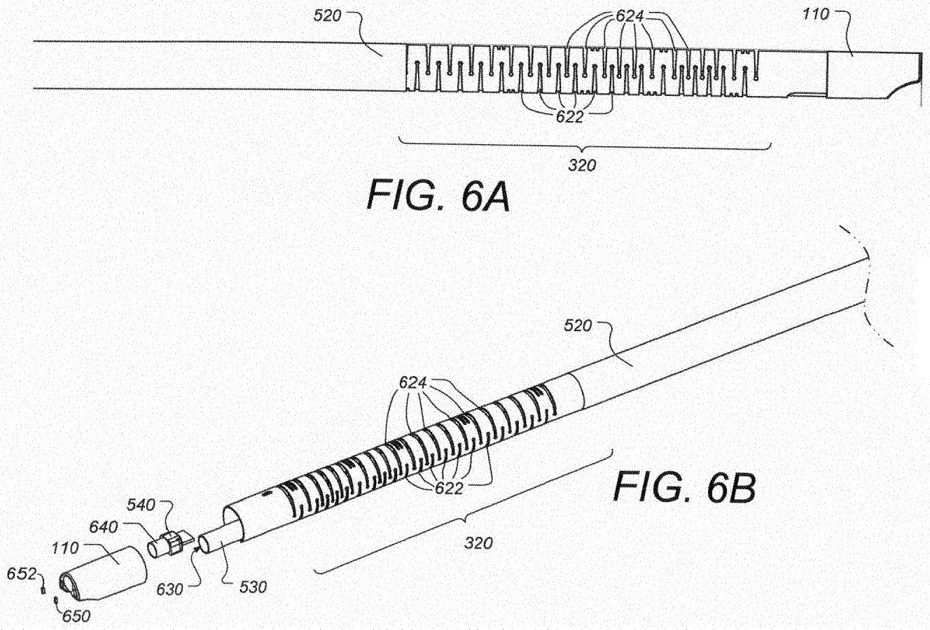

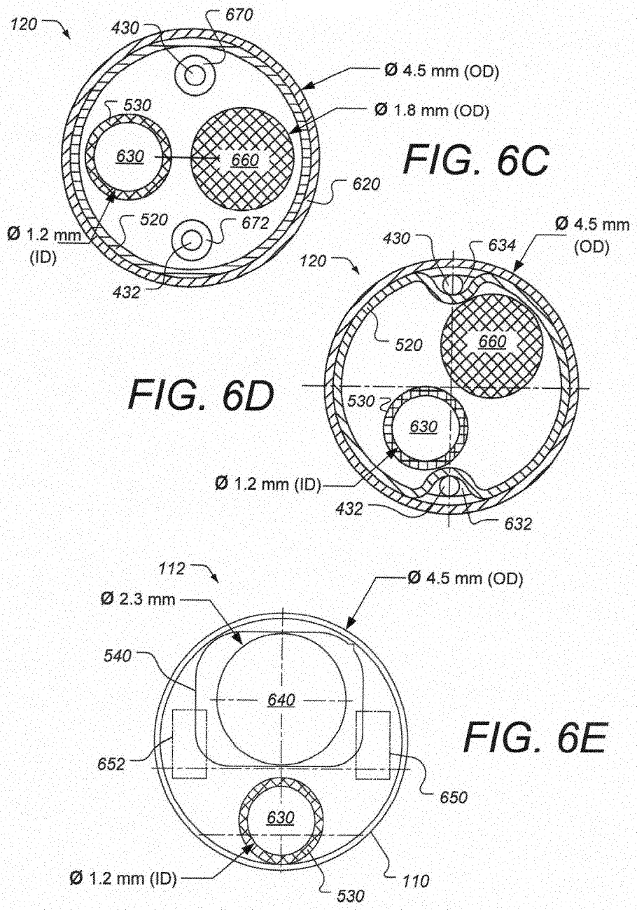

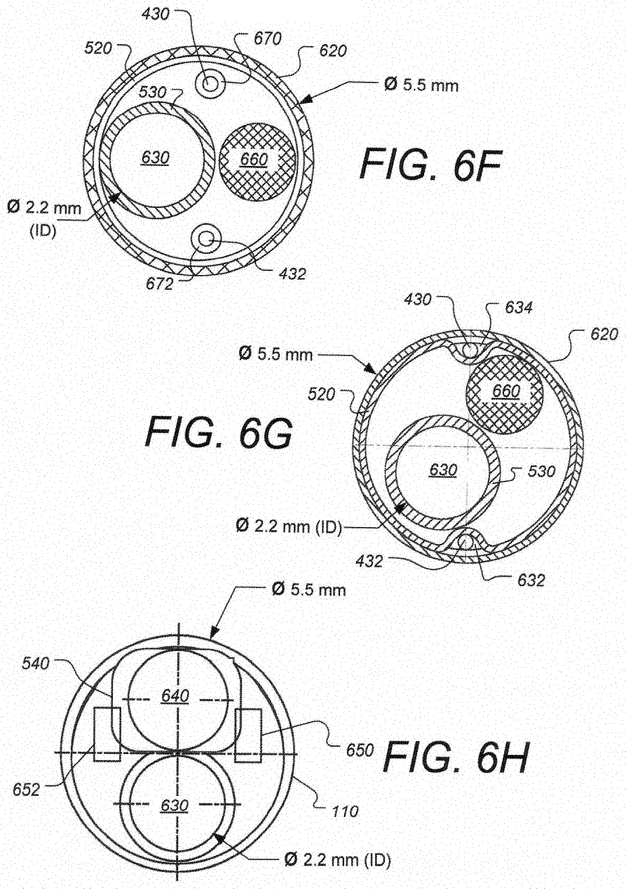

The surgical endoscope 100 includes an elongated cannula 120 with a distal tip 112 for inserting into a hollow organ or cavity of the body. According to some embodiments, a separate distal tip sub-assembly 110 is attached to the cannula 120. According to some embodiments, the distal tip 112 is less than 4.5 mm in diameter when no working channel or a narrower working channel 630 is included in the cannula (FIGS. 6C-E). According to some embodiments, for example when a wider working channel 630 is included, the distal tip diameter can be 5.5 mm (FIGS. 6F-H). For further details relating to a separate tip sub-assembly for a handheld endoscope, see said: U.S. Pat. No. 9,895,048 (hereinafter referred to as “the '048 patent”); U.S. Ser. No. 15/462,331 filed March 17, 2017 published as U.S. 2017-0188793 A1 (hereinafter the '331 application) and now U.S. Pat. No 10,524,636; and Intl Pat. App. No. PCT/US18/14880 filed Jan. 23, 2018 published as Intl Pub. No. WO/2018/136950 (hereinafter referred to as “the '880 application”). Sub-assembly 110 includes an imaging module and one or more LED 30 light sources for viewing the organ or cavity into which tip assembly 110 is inserted. The tip assembly 110 also includes one or more fluid ports.

According to some embodiments, the cannula 120 includes one or more fluid channels which are fluidly connected to fluid port 132 at fluid hub and connection assembly 130. Port 132 includes a Luer fitting to facilitate leak-free connection of port 132 with various medical fluid components. The fluid channels or lumens in cannula 120 are also connected to a distal facing fluid ports of tip assembly 110. According to some embodiments, wires running from the LED light sources and camera module in tip assembly 110 pass through a separate channel in cannula 120. According to some embodiments, the cannula 120 rotates about its long axis relative to the handle 140, as described in more detail below.

The endoscope 100 includes a handle portion 140 that is sized and shaped in a pistol-like fashion for easy grasping by the endoscope operator (e.g. doctor or other medical professional). A display module 150 is rotatably mounted on handle 140 via a bearing which can be a plain bearing made of plastic, and a rubber coated hinge. Also visible on handle 140 are image capture button 142 and power button 144. According to some embodiments handle 140 and display module 150 are configured to be re-usable and make up reusable portion 102. According to some embodiments, handle 140 is like handle 140 shown and described in the '048 patent, the '331 application, and the '880 application.

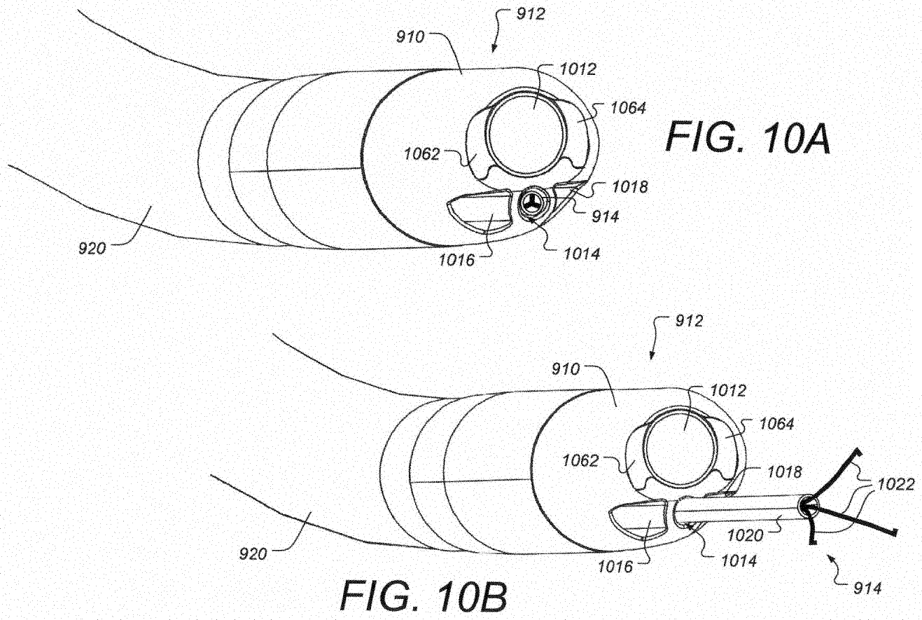

Single-use portion 104 includes steering actuation hub 170, fluid hub and connection assembly 130, cannula 120 and tip assembly 110. Single-use portion 104 is made at a relatively low-cost and is intended to be disposed of after a single-use. By assembling the tip, cannula and fluid hub assembled into a single-use portion, stringent decontamination and disinfection procedures as well as the risk of cross-contamination and hospital acquired diseases can be significantly lessened or avoided. As shown in FIG. 10, according to some embodiments, the disposable, single-use portion 104 is sterilized, for example, during production and is provided to the user in a sealed sterilized pouch 106, for ease of storage and handling. According to some embodiments, a fluid line (not shown) is also included in single use portion 104 and can be attached to port 132 and included in the same sterilized pouch 106.

FIG. 2 is a side view of a slim disposable steerable cannula, according to some embodiments. The distal end of cannula 120 is controllably steerable or bendable in both the upwards and downwards directions as shown. The distal end of cannula 120 is shown in a neutral, or un-deflected position 210. The position 212 is an example of an extreme upwards deflection of 210 degrees, for example, while position 214 is an extreme downwards deflection of 130 degrees, for example. The deflection is controlled by one or two levers on the proximal end that extend from actuation hub 170. In this example there are two levers: lower lever 220 and upper lever 230. The lower and upper levers 220 and 230 are fixed together and rotate about the central axis of hub 170 such that when lower lever 220 is pulled proximally toward dotted position 222 the upper lever 230 will move distally toward dotted position 232. Likewise, when the upper lever 230 is pulled proximally toward dotted position 234 the lower lever 220 will move distally toward dotted position 224. In this example, when the lower lever 220 is pulled proximally, the distal end of cannula 120 and distal tip 112 is bent upwards toward position 212 and when the upper lever 230 is pulled proximally, the distal end of cannula 120 and distal tip 112 is bent downward toward position 213. In other examples the deflection relationships can be reversed. In this example pulling either lever 220 or 230 proximally by 35 degrees will result in actuation or deflection of the distal end of cannula 120 and distal tip 112 to bend to the extreme positions 212 (210 degrees up) and 214 (130 degrees down). In other embodiments, other amounts of deflection can be configured for various amounts of lever actuation, and lever 230 can be omitted so that only a single lever 220 steers the bendable portion of cannula 120.

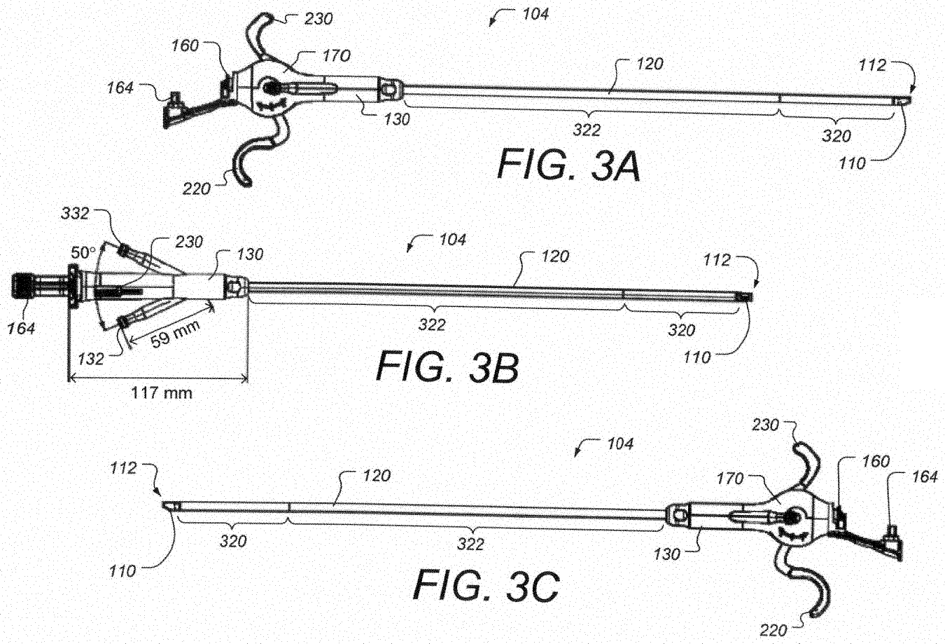

FIGS. 3A, 3B and 3C are right side, top and left side views, respectively, of a disposable steerable cannula, according to some embodiments. The cannula 120 is formed of a flexible portion 320 and a non-flexible portion 322. Also visible in FIGS. 3B and 3C is an optional second fluid and/or device port 332 on the left side of the fluid hub 130.

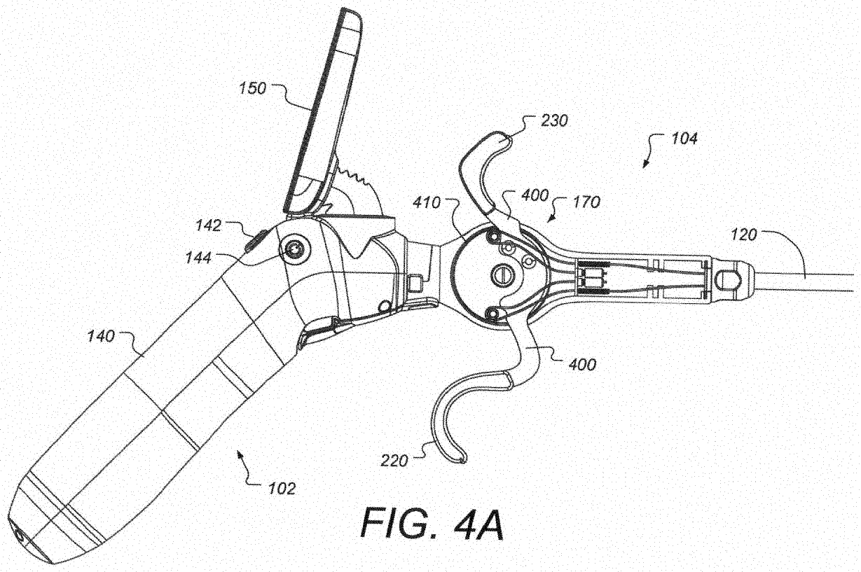

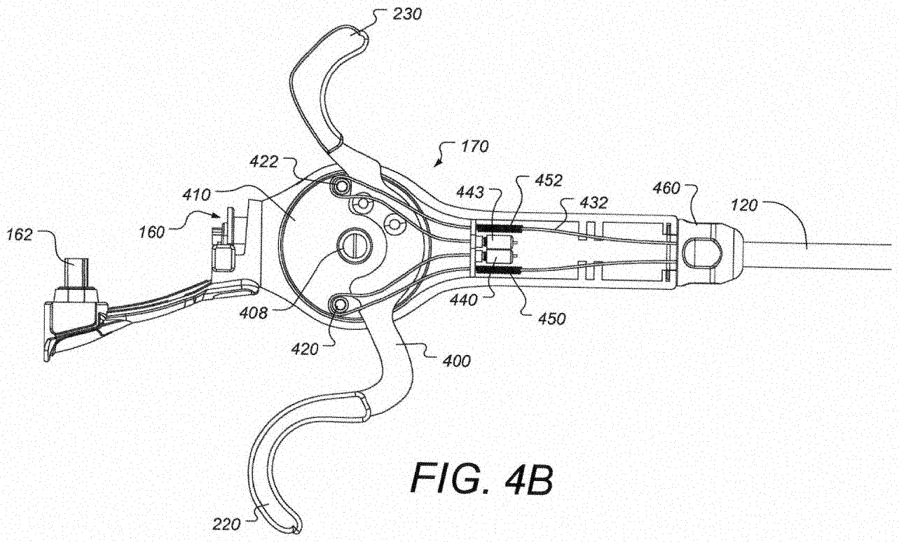

FIGS. 4A and 4B show further detail of a steering actuation hub of a portable endoscope having a slim disposable steerable cannula, according to some embodiments. The deflection is controlled by one or two levers that rotate a wheel 410 for pulling two cables 430 and 432 which actuate the deflection. FIG. 4A shows the proximal portion of disposable portion 104 mounted to the re-usable portion 102. FIG. 4B shows only parts of the disposable portion 104 for clarity.

The lower and upper levers 220 and 230 are both fixed to, or form an integral part of, lever arm 400. Lever arm 400 is configured to rotate wheel 410 about its central axis 408. Attached to wheel 410 are two small pulleys: lower pulley 420 and upper pulley 422. Lower cable 430 is fixed by nut 440. The lower cable 430 runs through lower pulley 420 and guide 450 and then continues along the length of cannula 120 where it is fixed near its distal end (not shown). The upper cable 432 runs through upper pulley 422 and guide 452 and then continues along the length of cannula 120 where it is fixed near its distal end (not shown). As can be seen, when the lower level 220 is moved proximally, the wheel 410 will rotate clockwise. This will cause the lower pulley 420 to pull-on the lower cable 430 while causing the upper pulley to slacken the upper cable 432. The distal end of cannula 120 is configured to cause an upward deflection when cable 430 is tightened and cable 432 is slackened. Likewise, when upper lever 230 is moved proximally, the wheel 410 will rotate in a counter-clockwise direction. This will cause the upper pulley 422 to pull-on the upper cable 432 while causing the lower pulley to slacken the lower cable 430. The distal end of cannula 120 is configured to cause a downward deflection when cable 432 is tightened and cable 430 is slackened. For this purpose and in this non-limiting example, cables 430 and 432 cross before reaching the distal end of cannula 120 so that lower cable 430 is attached to an upper part of tip 112 and upper cable 432 is attached to a lower part of tip 112. Because of such crossing, pulling the lower lever 220 in the proximal direction bends the cannula's distal tip upwardly and pulling upper lever 230 bends the cannula's distal tip downwardly. Such cable crossing can take place distally of the actuation hub. Alternatively, the cables can cross within hub 170, for example by having cable 430 runs through guide 452 and attach to an upper portion of cannula tip 112 and having cable 432 run through guide 450 and attach to a lower part of cannula tip 112. It has been found desirable in some medical procedures to bent distal tip 112 upward by using the more natural motion of pulling lower lever 220 in the proximal direction, as in pulling a gun trigger. Upward bending, particularly through a larger angle, uniquely assists some medical procedures, such as procedures in which it is desirable to view the neck of a bladder by bending the cannula tip so much that the field of view of the endoscope is back, in the proximal direction.

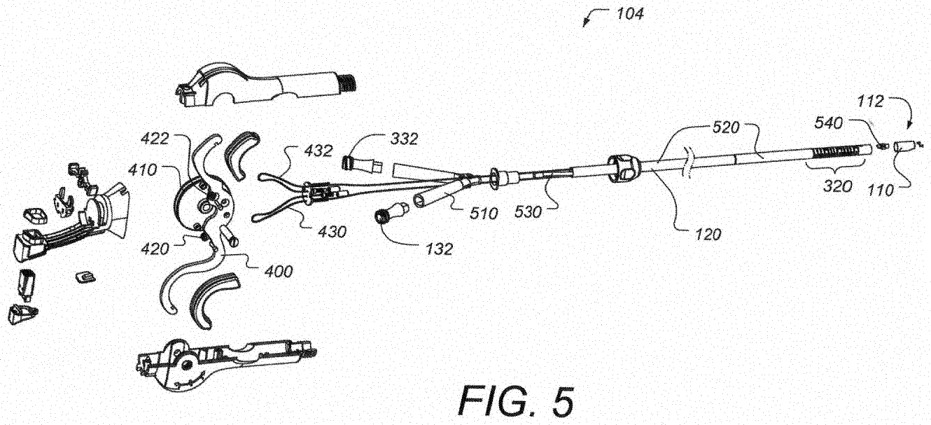

FIG. 5 is an exploded view illustrating various components of the disposable portion 104, according to some embodiments. Visible at the proximal end are the lever arm 400, wheel 410, pulleys 420 and 422, and upper and lower cables 432 and 430. Also visible are fluid/device conduits 510. Conduit 530 can be used as a device working channel and/or a fluid channel and is shown inserted in cannula 120 such that cannula 120 can rotate about its longitudinal axis relative to conduit 530 and thus relative to handle 140. This rotation can be accomplished by the user rotating by hand cannula 120 while holding handle 140. Rotation can be limited in angle, such as to roughly 90 degrees, to avoid undue twisting of the electrical and steering cables running from handle 140 toward the distal end of cannula 120. According to some embodiments, the cannula 120 is made of a steel tube 520 which has a series of notches cut in the flexible portion 320 that allow tube 520, and cannula 120 to bend upwards and downwards. At the distal end the flexible portion 320 of cannula 120 and tube 520 is shown distal tip piece into which fit camera module 540 and two LEDs.

FIGS. 6A and 6B are a side view and a perspective view illustrating further detail of a distal portion of an endoscope according to some embodiments. Lower notches 622 and upper notches 624 that alternate and allow for upward and downward bending of tube 520 at flexible portion 320, are shown in greater detail. Steering cables 430 and 432 connect to respective points at distal portions of the notched portions of cannula 120 to effect steering. In FIG. 6B the conduit 530 is shown that can form working channel 630. The camera module 540 includes a lens portion 640 and fits into tip piece 110, as do LEDs 650 and 652.

FIGS. 6C-6H are cross sections illustrating further details of a distal portion of an endoscope according to some embodiments. FIGS. 6C, 6D and 6E are cross sections of the cannula 120 and distal tip 110 where the outer diameter of the cannula 120 and distal tip are 4.5 mm and the inner diameter of the working channel 630 is 1.2 mm. FIG. 6C is a cross section of cannula 120 in a location proximal to the flexible portion 320. The steel tube 520 is shown in this case surrounded by a thin sealing outer layer 620 that is not shown in FIGS. 5, 6A and 6B for reasons of clarity. Outer layer 620 can be made of a material such as PTFE and in some cases can be installed around the entire outer surface of the cannula 120 and portions of tip 112 via heat-shrinking. The cables 430 and 432 are also shown, threaded through wire conduits 670 and 672 respectively. The conduits 670 and 672 can be made of stainless steel and run the length of the cannula 120 that is proximal to the flexible portion 320. Note that when comparing FIG. 6C with FIG. 4B, cable 430 crosses from the bottom cable in FIG. 4B to the upper cable in FIG. 6C and cable 432 crosses from the upper cable in FIG. 4B to the lower cable in FIG. 6C. The location where the cables cross over, according to some embodiments, in proximal to the cannula 120 such as within housing 460 shown in FIG. 4B.

FIG. 6D is a cross section of cannula 120 in the flexible portion 320. In this location, the wires 430 and 432 pass through conduits 634 and 632 respectively. Note that the distal ends of cables 430 and 432 are bonded within conduits 630 and 632, respectfully, at a location along tube 520 that is distal of the flexible portion 320 (and notches 622 and 624). Also visible in FIGS. 6C and 6D is electrical cable 660 which in these examples has an outer diameter of 1.8 mm. Cable 660 is used to transmit power and control information to the camera module 540 and LEDs in the distal tip and also to transmit image and video data from the camera module back towards the handle portion 140 (e.g. shown in FIGS. 1A and 1B). FIG. 6E is a cross section of the distal tip 112 showing the lens portion 640 and camera module 540. The location of LEDs 650 and 652 are also shown in dash-dotted outline. According to some embodiments, the working channel 630 can be used for fluid infusion and also can contain a small guidewire. FIGS. 6F, 6G and 6H are cross sections of the cannula 120 and distal tip 110 in an example where the outer diameter of the cannula 120 and distal tip are 5.5 mm and the inner diameter of the working channel 630 is 2.2 mm. In this example the various components and materials are like the corresponding components and materials shown in FIGS. 6C-6E.

Further details relating to flexible portion 320 of cannula 120 and further aspects of steering and bending cannulae are provided in co-pending patent application U.S. Ser. No. 15/856,077 filed Dec. 28, 2017, published as U.S. Pat. App. Publ. US 2019/0059699 on Feb. 28, 2019, and now U.S. Pat. No. 10,918,268, which is incorporated herein by reference.

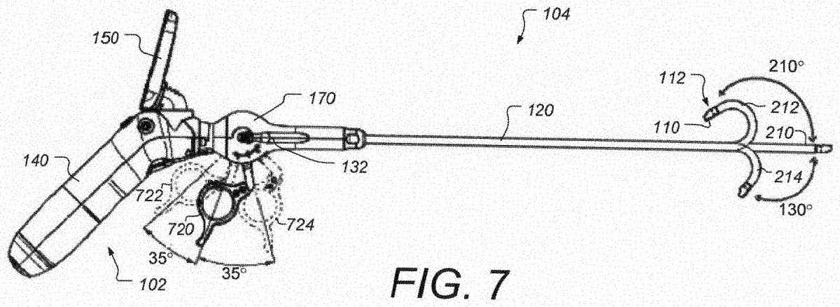

FIG. 7 is a side view of a slim disposable steerable cannula, according to some embodiments. The disposable portion 104 in this case is similar or identical in many respects as that described above except that the steering is controlled by a single lever 720 instead of two levers. The lever 720 is fixed to and rotates about the central axis of hub 170 such that when lever 720 is pulled proximally toward dotted position 722 the distal end of cannula 120 and distal tip 112 is bent upward towards position 212 and when the lever 720 is pushed distally toward dotted position 724, the distal end of cannula 120 and distal tip 112 is bent downward towards position 213. In other examples the deflection relationships can be reversed. In this example pulling or pushing lever 720 by 35 degrees will result in actuation or deflection of the distal end of cannula 120 and distal tip 112 to bend to the extreme positions 212 (210 degrees up) and 214 (130 degrees down). In other embodiments, other amounts of deflection can be configured for various amounts of lever actuation. Cannula 12o is configured for rotation about its longitudinal axis relative to handle 140, as indicated by the arrow at the distal portion of hub 170.

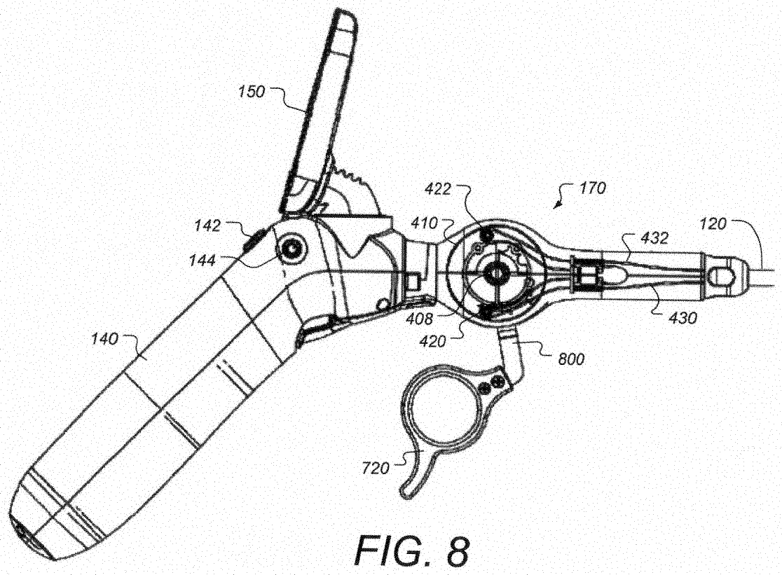

FIG. 8 shows further detail of an steering actuation hub of a portable endoscope having a slim disposable steerable cannula, according to some embodiments. The deflection is controlled by lever 720, which is fixed to arm 800 to rotate wheel 410. The rotation of wheel 410 pulls on or slackens cables 430 and 432 which actuate the deflection.

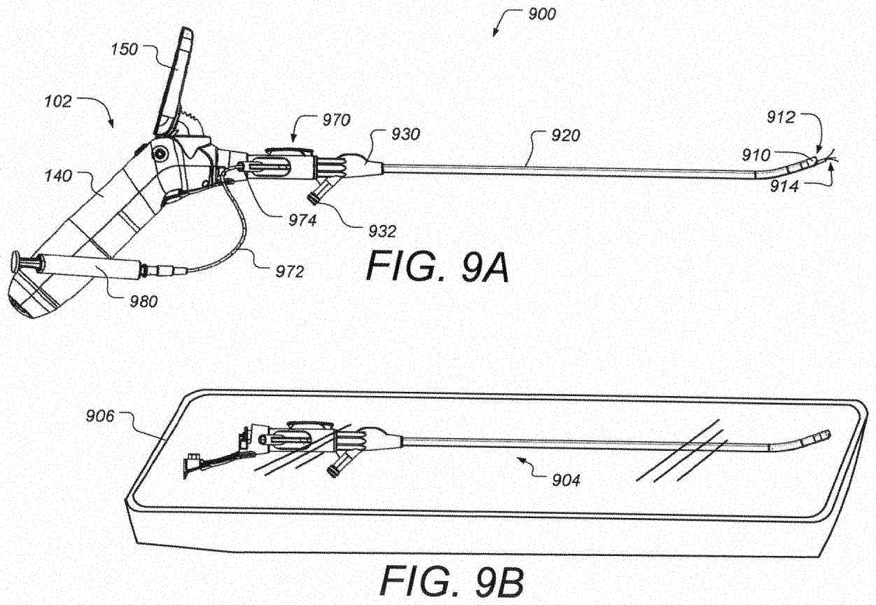

FIGS. 9A and 9B are a right side view a perspective view, respectively, of a handheld surgical endoscope having an integrated grasping tool, according to some embodiments. The surgical endoscope 900 includes an elongated cannula 920 with a distal tip 912 for inserting into a hollow organ or cavity of the body. A grasper 914 passes trough a dedicated lumen in cannula 920. The grasper 914 can be extended to protrude distally from distal tip 912 as shown. The grasper 914 can be attached to or formed as an integral part of a solid or hollow tube that can be actuated with actuation hub 970. In cases where grasper 914 includes a hollow tube, it can be in fluid communication with fluid line 972, which in turn is connected to syringe 980 (or other fluid dispensing device).

According to some embodiments, a separate tip sub-assembly 910 is attached to the cannula 920 which can be made from an extruded material. For further details relating to a separate tip sub-assembly for a handheld endoscope, see the '048 patent, the '331 application, and the '880 application. The tip assembly 910 includes an imaging module and one or more LED light sources for viewing the organ or cavity into which it is inserted. The tip assembly 910 also includes one or more fluid ports. The distal end of the cannula 920 can also be slightly bent as shown. According to some embodiments, a bend of about 15 degrees has been found to be suitable for many applications, but using other angles in alternative embodiments is not excluded.

According to some embodiments, the cannula 920 includes one or more fluid channels which are fluidly connected to fluid port 932 at fluid hub and connection assembly 930. Port 932 includes a Luer fitting to facilitate leak-free connection of port 932 with various medical fluid components. The fluid channels or lumens in cannula 920 are also connected to a distal facing fluid ports (orifice or ports 1016 and 1018 shown in FIGS. 10A, and 10B) of tip assembly 910. According to some embodiments, wires running from the LED light sources and camera module in tip assembly 910 pass through a separate channel in cannula 920.

The endoscope 900 includes a handle portion 140 that is like handle portion 140 shown and described above and in the '048 patent and the '880 application. Single-use portion 904 includes needle actuation hub 970, fluid hub and connection assembly 930, cannula 920 and tip assembly 910. Single-use portion 104 is made at a relatively low-cost and is intended to be disposed of after a single-use. By making the tip, cannula, and fluid hub all single-use, stringent decontamination and disinfection procedures as well as the risk of cross-contamination and hospital acquired diseases can be significantly lessened or avoided. According to some embodiments the disposable, single-use portion 904 is sterilized, for example, during production and is provided to the user in a sealed sterilized pouch 906, for ease of storage and handling as shown in FIG. 9B. The camera module in the tip assembly can have a wide angle of view, such as 140 degrees in this example. According to some embodiments, the fluid line 972 is also included in single use portion 904 and can be attached to hub 970 and included in the same sterilized pouch 906. According to some embodiments, the surgical endoscope is configured to allow cannula 920 to rotate about its longitudinal axis. For further details of how to configure the hub 130 to allow rotation of the cannula, see the '048 patent, the '331 application, and the 880 application.

FIGS. 10A and 10B are perspective views of distal tip 912 and show aspects of the grasper actuation, according to some embodiments. FIG. 10A shows tip 912 with grasper 914 in the retracted position while FIG. 10B shows tip 912 with grasper 914 is in the extended position. Note that while in the retracted position, the claws of grasper 914 are fully recessed within grasper port 1014 of tip assembly 910 and there is no risk of a sharps injury from the tip of grasper 914. Also visible in FIGS. 10A and 10B are camera lens dust cover 1012, two light-guide lenses 1062 and 1064 (for LED light sources) and distal fluid ports 1016 and 1018. Distal fluid ports 1016 and 1018 are provided to allow for fluid communication with the fluid lumina of cannula 920 (not shown). In this example, each of fluid ports 1016 and 1018 can have a cross sectional area of about 1.6 mm². Note that port 932, the cannula lumina and distal fluid ports 1016 and 1018 can be configured to provide fluid in-flow (i.e. flowing fluid out of the endoscope and into the patient's organ or cavity and/or fluid out-flow (i.e. flowing fluid out of the patient's organ or cavity and into the endoscope).

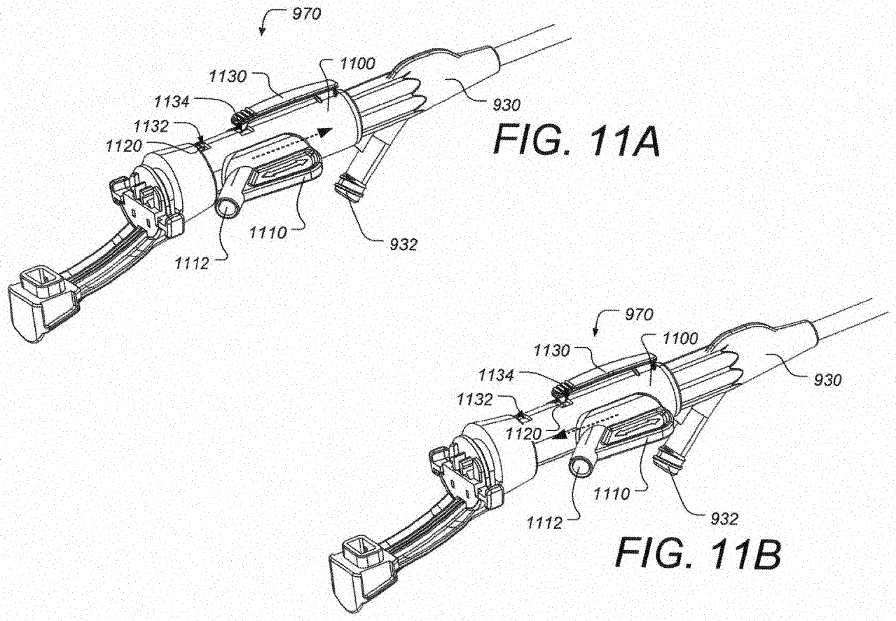

FIGS. 11A and 11B are perspective views showing aspects of grasper actuation for a handheld surgical endoscope, according to some embodiments. FIG. 11A shows grasper actuation hub 970 when grasper 914 is in the retracted position as shown in FIG. 10A, while FIG. 11B shows hub 970 when grasper 914 is in the extended position as shown in FIG. 10B. Hub 970 includes an outer housing 1100 through which are formed two windows, proximal window 1132 and distal window 1134. A lock release button 1130 extends from the housing 1100 and includes a inwardly protruding tab that aligns with distal window 1134. Actuation tab 1110 is moveable relative to the hub housing 1100. Moving with tab 1110 is fluid port 1112 that is in fluid communication with fluid line 972 (not shown), spring tab 1120 and grasper 914 (not shown). For further detail of the movable portions of hub 970, see FIG. 7B of the '331 application.

FIGS. 10A and 11A show the grasper 914 in the retracted position. In this positions, as mentioned the claws of grasper 914 are retracted within the grasper port 1014. In this retracted position, the distal tip 912 of the endoscope can be inserted into the organ and/or tissue of interest. When the operator observes on the display a target tissue that he/she wishes to manipulate, the actuation tab 1110 is pushed distally (as shown by the dotted arrow in FIG. 10A) until it is in the position shown in FIG. 10B. Using the display 150, the user then maneuvers the claws 1022 to be in a position surrounding the target tissue. In order to close the claws 1022, the actuation tab is moved proximally (as shown with the dotted arrow in FIG. 10B). It has been found effective in some cases to slide the endoscope distally at the same time as the actuation tab is moved proximally, so that the claws 1022 can remain in the same position relative to the target tissue. When the claws 1022 begin to engage with the distal rim of grasper port 1014, the claws 1022 begin to close upon each other. As the grasper is further retracted into the grasper port 1014, the grasping force of the claws 1022 increases. When the claws have sufficiently grasped the target tissue, the operator can then move the distal tip as appropriate to carry out the intended procedure.

The positions of the levers described above relative to the longitudinal axis of the cannula correspond to respective degrees of bending of the distal portion of the cannula, in some embodiments. A lever need not be subjected to additional force to keep it in position once the distal portion of the cannula has bent to a desired degree, except for any force that might be needed to overcome any tendency of the distal portion of the cannula to spring back toward an orientation along the longitudinal axis.

In some embodiments some, most, or all of the length of the cannula can be made of a material that is sufficiently flexible to allow the cannula to conform at least to some extent to curved body cavities or passages as it is inserted in the patient.

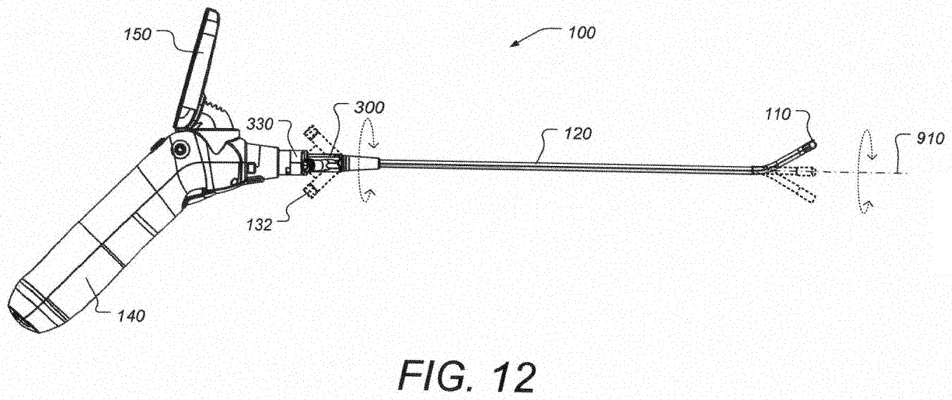

FIG. 12, which is like FIG. 9A of the '048 patent, illustrates rotation of the cannula 120 about a longitudinal axis, relative to handle 140. As disclosed in the '048 patent in connection with FIG. 9A therein, cannula 120 along with the distal tip 110 and fluid hub 300, is rotatable about main axis 910. The portion of the assembly that rotates with cannula 120 includes fluid port 132, fluid hub 300 and an inner tube that forms the inner portion of sleeve bearing 330. Rotation of cannula 120 and hub 300 can be limited so that the internal electrical cable and the steering cables do not undergo undue stress from twisting. In one example starting from a “neutral” position shown in solid lines in FIG. 12, cannula 120 can be rotated 30 about 180 degrees in in either direction (i.e., clockwise or counterclockwise) and in another example rotation can be limited to roughly 90 degrees in each direction. According to some other embodiments, an asymmetrical rotation pattern can be implemented in sleeve bearing 330 such as 270 degrees in one direction and 90 degrees in another direction. Many other combinations can be implemented, to improve ergonomics for various situations (i.e., various users, types of procedures, and patient anatomy variations). FIGS. 5 and 7 include arrows indicative of rotation of cannula 120 relative to pistol-grip handle 140 about a longitudinal axis along which cannula 120 extends.

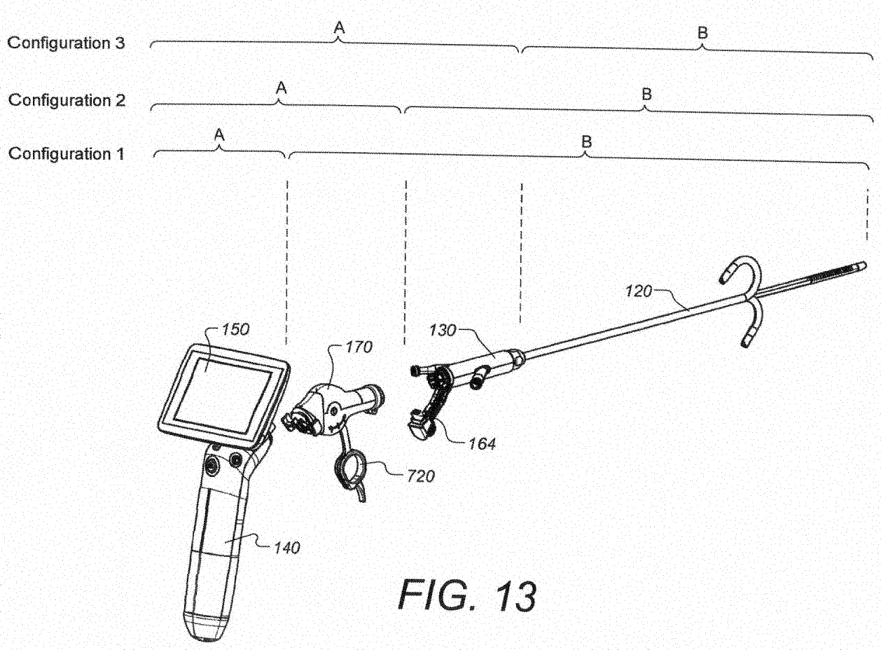

FIGS. 13-21 illustrate endoscopes and components thereof that can be used in any of the other endoscopes disclosed in this patent specification, and are discussed in detail below.

FIG. 13 shows in perspective three subassemblies of an endoscope and how they can be configured as single-use disposable portions and multiple-use reusable portions in four different configurations 1-4 of multiple-use and single-use portions, according to some embodiments. While FIG. 13 illustrates the use of two finger levers 230 and 720 (like levers 220 and 230 220 in FIG. 2), the endoscope of FIG. 13 can alternatively and preferably use only a finger lever 720, as in the example of FIG. 7 where only a single lever 720 steers cannula 120. The illustrated sub-assemblies can be supplied to a user such as a health professional in one or four different configurations, as indicated by the brackets numbered 1-4 in FIG. 13: (1) a multiple-use portion A comprising handle 140 and display module 150 attached to each other and a single-use portion B comprising steering actuation hub 170, fluid hub 170 and cannula 120 attached to each other, (2) a multiple-use portion A comprising handle 140, display module 150 and steering actuation hub 130 attached to each other and a single-use portion B comprising fluid hub 130 and cannula 120 attached to each other, (3) a multiple-use portion A comprising handle 130, display module 150, steering actuation hub 170 and fluid hub 130 attached to each other and a single-use portion B comprising cannula 120, and (4) each of sun-assemblies 140, 150, 170, 130 and 120 supplied as a separate unit, allowing any of the subassemblies or combination of subassemblies to be treated as multiple-use or single-use portions. These four configurations can be used with any of the endoscopes disclosed in this patent specification.

Portions A and B can be supplied to a user at the same time or at different times. The components that make up portion A can be supplied attached to each other permanently, i.e., in a way keeping the user from conveniently detaching them from each other, or they can be supplied to a user attached releasably so the user can detach them from each other by hand. Likewise, the components that make up portion B can be supplied attached to each other permanently or releasably. An advantage of permanent attachment over a releasable attachment is saving a user's time in assembling the portions into an endoscope for use in a patient procedure and helping avoid assembling errors. Toe assemble a complete endoscope, the multiple-use portion A and the single-use portion B can be attached to each other and detached from each other by hand, without a need for tools.

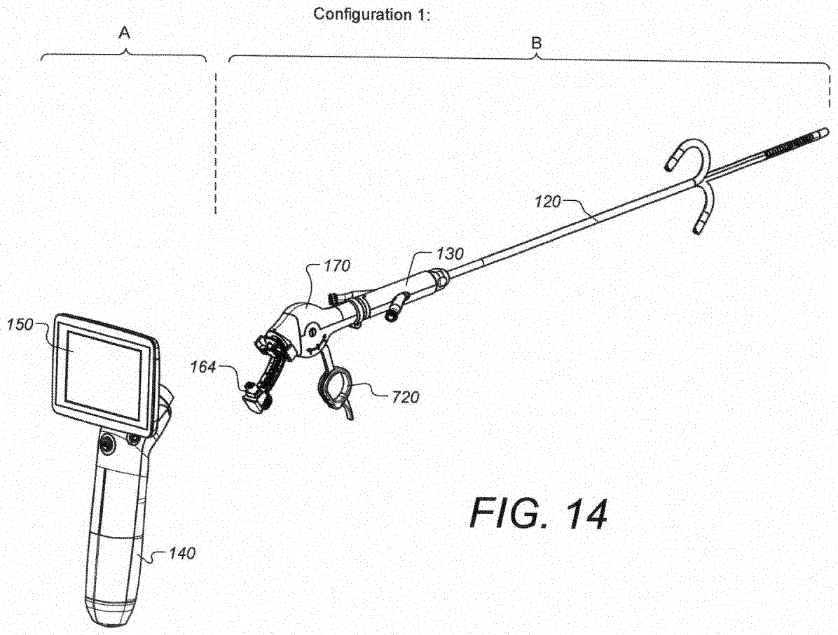

FIG. 14 illustrates configuration 1 identified in FIG. 13. The multiple-use portion A and the single-use portion B can be supplied in respective sterile packaging and unwrapped just before a patient procedure and assembled into an endoscope as described above, or only portion B can be so packaged. Or, the user may have a supply of packaged single use portions and can select one to assemble with a previously used multiple-use portion that has been cleaned. One benefit of this configuration 1 is that the single-use portion B, which contacts the patient can be disposed after a patient procedure while the multiple-use portion, which is away from the patient, can be kept for another patient procedure. Simpler sterilization or cleaning can be sufficient for portion A between patients. The multiple-use portion A in this configuration typically is significantly more expensive than the single-use portion B and re-using it promotes efficiency and lowers the cost of a patient procedure.

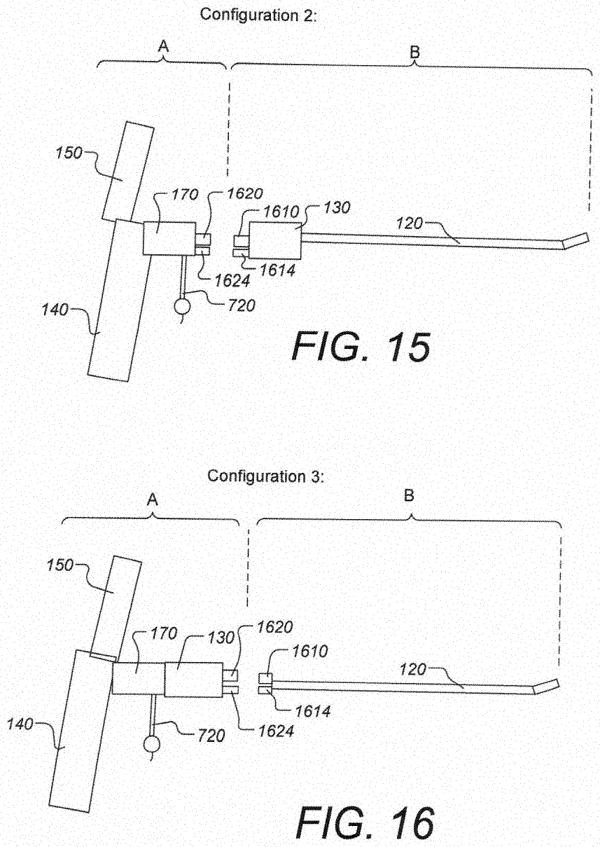

FIG. 15 schematically illustrates configuration 2 identified in FIG. 13. In this case, multiple-use portion A comprises handle 140, display module 150 and steering actuation hub 170 assembled together and supplied to a user while attached to each other, preferably permanently but alternatively releasably, at one time or at different times. In this configuration, the electrical and mechanical connection between handle 140 and steering actuation hub 170 can be as in FIGS. 1, 7 and 12. Alternatively, as this can be done when manufacturing, the mechanical connection can be by securing hub 170 to handle for example by a friction fit or by adhesives and establishing an electrical connection by internal cables replacing the function of contacts 164 and 166. One advantage of this configuration 2 is that the electrical connection between steering actuation hub 170 and handle 140 can be completed when unit A is manufactured, saving the user's time when assembling the endoscope for a patient procedure and avoiding possible assembly errors. In this configuration, electrical coupling of the single-use portion B to the multiple-use portion A can use connectors 1510 at the proximal end of portion B releasably mating with connectors 1520 at the distal end of hub 170. Mechanical coupling can be achieved using connectors 1514 and 1524 at the facing portions of steering actuation hub 170 and fluid hub 130 to releasably secure them to each other and also to releasably connect steering cables in steering actuation hub 170 to steering cables that extent distally through fluid hub 130 and cannula 120. For example, the mechanical couplers can be a friction-fitting sleeve at one side and a knob at the other side of the interface of the two hubs, where the knob snaps into the sleeve to assemble the endoscope, and the steering cable connection can be similarly implemented from portion A to portion B can be similarly implemented by a snap-in connection from a distal end of a cable in portion A to a proximal end of a cable from portion B.

FIG. 16 schematically illustrates configuration 3 identified in FIG. 13. In this case, single-use portion B comprises cannula 120 while multiple-use portion A comprises the remaining components of the endoscope supplied as assembled together permanently or releasably. In this configuration, releasably mating electrical contacts 1610 and 1620 are provides at the distal end of fluid hub 130 and at the proximal portion of cannula 120, and steering cables 430, 432 (FIGS. 5 and 7) extend distally to the distal end of fluid hub 130 and end in mechanical connectors 1624 that releasably mate with mechanical connectors 1614 at the proximal end of the steering cable that extend distally into cannula 120, for example by using snap-in connectors.

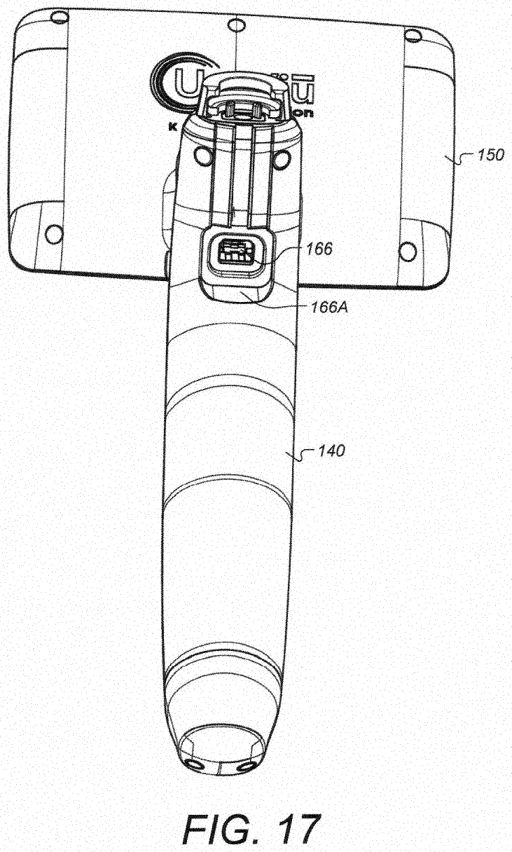

FIG. 17 illustrates a handle 140′ with an electrical contact 166 at its underside that is at the bottom of in a ramp-like depression 166A. Depression 166A slopes or tapers or steps inwardly toward connector 166 to facilitate orienting connector 164 toward and into engagement with connector 166 when assembling any of the endoscope examples described in this patent specification. As seen in FIG. 17, the entry into depression 166a in handle 140 around is made much larger than electrical connector 166 and tapers or steps down in size toward connector 166 to guide the connector 164 toward a correct position for mating with connector 166.

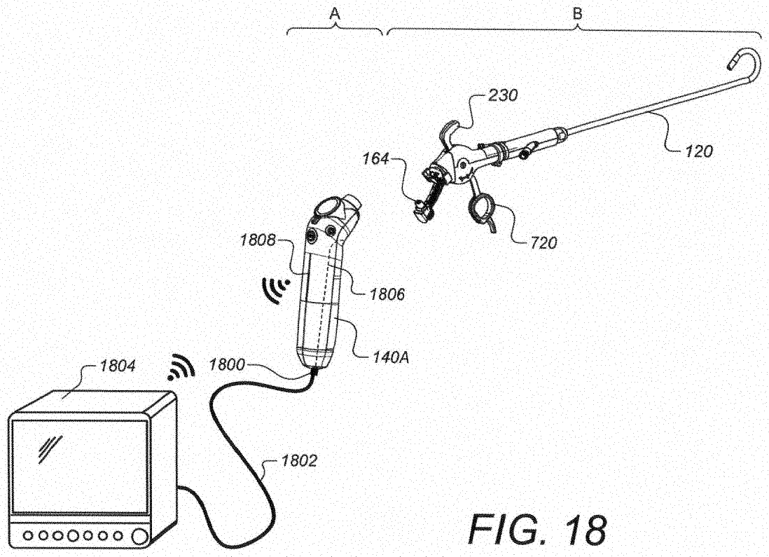

FIG. 18 illustrates an endoscope that omits an integral display module 150 and has a simpler and less expensive handle 140A connected to an external processor and display 1804. The connection can be by cable 1802 that plugs into electrical connector 1800 in handle 140A that can be a standard USB port, which in turn connects to connector 164 through internal wires 1806. Alternatively, handle 140A can include a WiFi or other wireless transmit/receive board 1808 that communicates wirelessly with processing/display unit 1804 that can serve the functions described above for display unit 150 and electronics in handle 140. This configuration can be used for any of the endoscopes disclosed in this patent specification.

While FIG. 18 illustrates two finger-operated levers 720 and 230, lever 230 can be omitted so that only lever 720 is used to steer cannula 120, as illustrated in FIG. 7. The cost of handle 140A in this example is sufficiently low to make handle 140A single-use, disposable after a patient procedure. In the example of FIG. 18, handle 140A can be supplied as a separate unit so that a single unit B of one or several available sizes can be attached to it, or the entire endoscope seen in FIG. 18 can be supplied as fully assembled, with portions A and B attached to each other permanently or releasably, preferably in a sterile pouch. In this example, entire endoscope can be disposed after a single use but processing and display unit 1804 can be kept and used for multiple patient procedures.

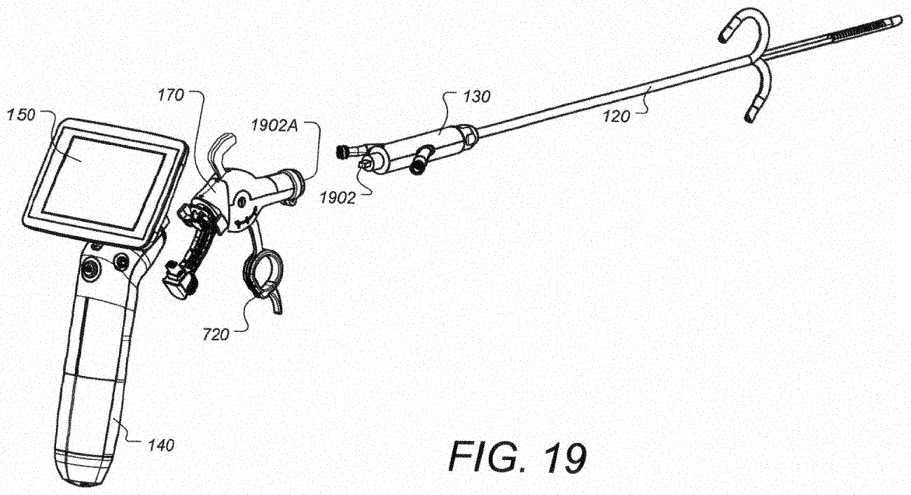

FIG. 19 illustrates in perspective an endoscope that has a modified connector 1902 at a proximal portion of a hub 130 and a mating connector 1902A at a distal end of steering hub 170. These two connectors mate to establish an electrical connection between the imaging and lighting module at the tip of cannula 120 and the electronics in handle 140 and display 150. In this example, cannula 120 can be mounted for rotation about its longitudinal axis relative to hub 130 and thus relative to hub 170 and handle 140. Additionally, fluid hum 130 can be mounted for rotation about the longitudinal axis relative to steering hub 170, and thus relative to handle 140 as well, as in the case of the endoscope of FIG. 12, by making the electrical and mechanical connectors at the distal side of steering hub 170 rotatable relative to the electrical and mechanical connectors at the proximal side of fluid hub 130.

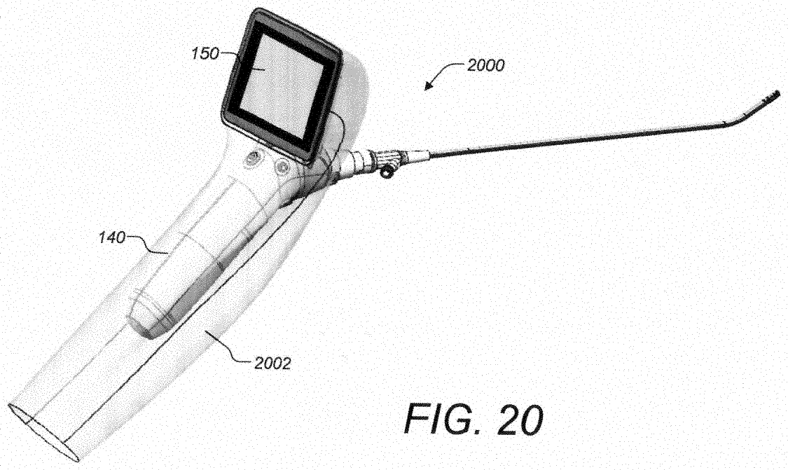

FIG. 20 illustrates in perspective an endoscope 2000, which can be any of the endoscopes described in this patent specification, equipped with a sterile robe 2002 that covers handle 140 and display 150 and may cover a proximal part of a hub extending distally from handle 140 through an opening 2004 at a distal face of robe 2002.

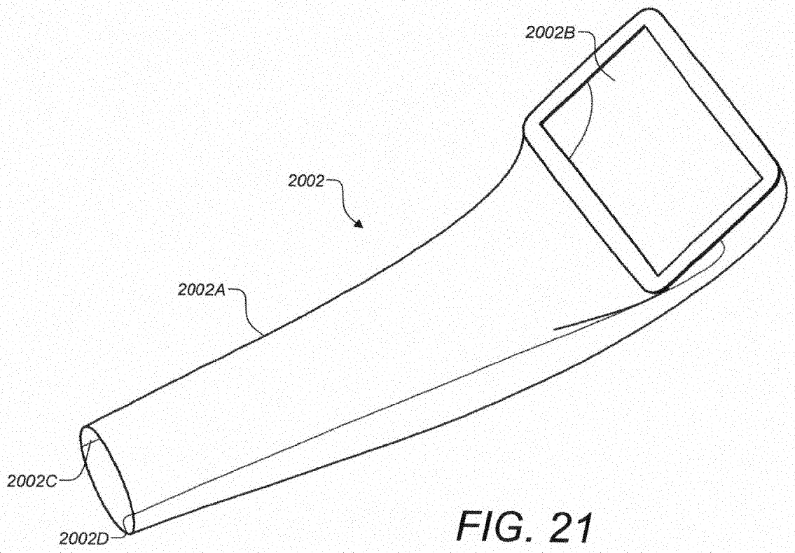

FIG. 21 illustrate robe 2002 in perspective. Robe 2002 comprises a lower portion 2002A shaped as a sleeve that loosely fits over handle 140 and an upper portion 2002B that fits over display 150 and has a transparent window that matches in size and fits closely the screen of display 150. Robe 2002 preferably is made of soft plastic material that is sufficiently stretchable to fit over handle 140 and display 150 as shows and to have its window closely fit and match in size the screen of display 150. The window of robe 2002 should be transparent, and the remainder of robe 2002 may but need not be transparent. Robe 2002 can be a single-piece robe or it can be made in two pieces that overlap at the lines numbered 2002C and 2002D to make easier fitting them over handle 140 and display 150. In this case, the material of robe 2002 can be selected such that the overlapped portions cling to each other to hold robe 2002 in place.

FIG. 20 shows an endoscope 2000 that does not have a lever for steering control but sleeve 2002 cab be used with any of the other endoscopes shows in this patent specification, including those with steerable cannula tips and one or two levers to control steering, by making opening 2004 in robe 2002 the appropriate shape and size to accommodate a steering control hub 170 and one or more levers such as 220, 230 and 720 used in other endoscopes.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

1. An endoscope for procedures in a patient, comprising:
a cannula having a bendable distal portion and an imaging module distal from said bendable portion;
a fluid hub, a steering hub, a handle, and a steering lever, wherein:
said fluid hub has a distal end configured to couple with a proximal end of said cannula;
said steering hub has a distal end configured to couple with a proximal end of said fluid hub;
said handle has a distal end configured to couple with a proximal end of said fluid hub; and
said steering lever is operatively coupled to said bendable portion of the cannula and is configured to respond to manual manipulation of the lever to bend said bendable portion of the canula in at least two differed angular direction and to a selected degree in each of said angular directions;
wherein said cannula, fluid hub, steering hub, and handle are in a selected one of first through fourth configurations as supplied to a user wherein:
(i) in the first configuration said cannula, fluid hub, and steering hub are assembled connected to each other as a first single-use portion and said first single use portion is in a first sterile pouch but said handle comprises a first multiple-use portion that is outside the first sterile pouch;
(ii) in the second configuration said cannula and fluid hub are assembled connected to each other in a second single-use portion and said second single use portion is in a second sterile pouch but said steering hub and said handle are assembled connected to each other as a second multiple-use portion that is outside the second sterile pouch;
(iii) in the third configuration said cannula comprises a third single-use portion and said third single use portion is in a third sterile pouch but said fluid hub, steering hub and handle are assembled together as a third multiple-use portion that is outside the third sterile pouch; and
(iv) in the fourth configuration, said cannula, fluid hub, steering hub, and handle are separate and unconnected to each other as supplied to a user; and
wherein said cannula, fluid hub, steering hub, and handle are configured for assembly into said endoscope for a procedure in a patient.

2. The endoscope of claim 1, in which said cannula, fluid hub, steering hub, and handle are supplied to a user in said first configuration.

3. The endoscope of claim 1, in which said cannula, fluid hub, steering hub, and handle are supplied to a user in said second configuration.

4. The endoscope of claim 1, in which said cannula, fluid hub, steering hub, and handle are supplied to a user in said third configuration.

5. The endoscope of claim 1, in which said cannula, fluid hub, steering hub, and handle are supplied to a user in said fourth configuration.

6. The endoscope of claim 1, in which said cannula is configured for rotation relative to the handle when said cannula, fluid hub, steering hub, and handle are assembled with each other for form said endoscope.

7. The endoscope of claim 1, further including a display integrally mounted on said handle and configured to display images taken with said imaging module and said handle comprises internal circuits configured to process image data supplied by said imaging module into images displayed on said display.

8. The endoscope of claim 7, further including a flexible and stretchable sterile robe into which said display and handle fit, said robe including a transparent window matching a screen of said display.

9. The endoscope of claim 1, further including a processing and display assembly remote from said handle and electrically coupled therewith via a cable to receive image data from said imaging module and process said imaging date into images for display and display said images.

10. The endoscope of claim 1, further including a display remote from said handle and electrically coupled therewith via a cable to receive image data from said imaging module.

11. The endoscope of claim 1, in which said steering hub and fluid hub are configured for rotation relative to each other about a longitudinal axis of said cannula when the cannula, fluid hub, steering hub, and handle are assembled to form said endoscope.

12. The endoscope of claim 1, further comprising a display supported by and mounted on said handle and having a screen to display images provided by said imaging module, and a sterile robe configured to fit over said handle and display and having a transparent window covering said screen.

13. The endoscope of claim 1, in which said bendable portion of the cannula is configured to bend in one of said angular direction through a maximum that is less than a maximum through which said bendable portion is configured to bend in the other of said angular directions.

14. The endoscope of claim 1, in which said steering hub for rotation comprises an internal wheel and further including cables secured at one end to said wheel and at the other end to said bendable portion of the cannula.

15. An integrated, single use endoscope comprising:
a cannula having at least one lumen extending along a length of the cannula, a bendable distal portion, and an electronic imaging module at a distal portion of the cannula;
a fluid hub permanently connected operatively to a proximal portion of the cannula and having at least one port in fluid flow communication with said lumen;
a handle permanently connected operatively to a proximal portion of the fluid hub;
a steering control operatively connected to said bendable portion of the cannula and manually operable to selectively bend said bendable portion through selected angles in at least two angular directions; and
an electronic port operative coupled with said imaging module and configured to receive power and commands from a location remote from the endoscope and to provide to said remote location image date generated by said imaging module;
wherein processing of said image date into images for display takes place primarily outside of said endoscope, and said endoscope is configured for only a single use in a patient procedure.

16. The endoscope of claim 15, in which said endoscope is operatively coupled with said remote location via a cable external to the endoscope.

17. The endoscope of claim 16, in which said endoscope is operatively coupled with said remote location wirelessly.

18. A method comprising:
providing a cannula having an internal lumen and a bendable distal portion with an imaging module distal from said bendable portion, a fluid hub proximal to the cannula, a steering hub proximal to the fluid hub, a handle proximal to the steering hub, an image display integral with said handle, and a steering lever:
selectively bending the bendable portion of the cannula in at least two angular directions and though selected angles in said angular directions by manually operating said steering lever;
selectively rotating at least one of the cannula and the fluid hub relative to the handle about a long axis of the cannula; and
producing image data with said imaging module, supplying the image data to said handle and display for processing the image data into display images and displaying said images.

19. The method of claim 18, in which said rotating comprises rotating the cannula relative to the housing.

20. The method of claim 18, in which said rotating comprises rotating both the cannula and the fluid hub relative to the housing.
